(19) United States
(12) Patent Application Publication
(10) Pub. No.: US 2025/0114104 A1
(43) Pub. Date: Apr. 10, 2025

(54) PATELLA IMPACTION DEVICE AND PATELLA PREPARATION SET

(71) Applicant: Aesculap AG, Tuttlingen (DE)

(72) Inventor: Eliott Snauwaert, Chaumont (FR)

(21) Appl. No.: 18/901,221

(22) Filed: Sep. 30, 2024

(30) Foreign Application Priority Data
Oct. 5, 2023 (EP) 23201766.5

(51) Int. Cl. A61B 17/16; A61B 17/17

(52) U.S. Cl. CPC A61B 17/1677; A61B 17/1767

(57) ABSTRACT

A patella impaction device includes a first clamp configured to be pressed along a pressure axis and onto a dome-shaped surface of a patella implant to be connected to a resected patella. The first clamp includes a shell-shaped clamping element mounted on a holding structure of the first clamp in a floating manner and allowing the clamping element to move at least along one axis of movement within a plane extending perpendicular to the pressure axis. The patella impaction device can be a component of a patella preparation set.

### CROSS-REFERENCE TO RELATED APPLICATION(S)

This application claims priority under 35 U.S.C. § 119 to European Application no. 23 201 766.5, filed on Oct. 5, 2023, the content of which is incorporated by reference herein in its entirety.

### FIELD

The invention relates to a patella impaction device, comprising a first clamp configured to be pressed along a pressure axis and onto a dome-shaped surface of a patella implant to be connected to a resected patella.

### BACKGROUND

During a total knee replacement or total knee arthroplasty (“TKA”) surgery, the posterior portion of a patella may be resected for implanting with a patella implant. The step of resecting the patella requires the use of a patella clamping device for retaining or holding the patella during cutting. US 2006/0161165 A1 proposes to provide a pincer-like structure with two mutually movable handle parts, one handle part being connected to a first clamping section engaging the skin/subcutaneous tissues overlying the anterior of a patella and the other handle part being connected to a second clamping section engaging the posterior of a patella, thus providing a clamping action along an antero-posterior axis of the patella.

Patella impaction devices are also known in the art and used for pressing a patella implant against a resected patella during a hardening process of a cement which connects the patella implant to the resected patella.

The choice of the size and the geometry of a specific patella implant depends on the size and geometry of the knee of the patient.

One object of the present invention is to provide a patella impaction device and a patella preparation set which allow for a reliable impaction of patella implants having a different geometry.

### SUMMARY

The patella impaction device comprises a shell-shaped clamping element which is mounted on a holding structure of the first clamp in a floating manner such that the clamping element is movable at least along one axis of movement within a plane extending perpendicular to the pressure axis. This movement allows for an alignment of a peak point axis of a specific patella implant with a corresponding center or lowest point of the shell-shaped clamping element without having to adjust the position of the holding structure.

In one embodiment, the clamping element is movable at least along a medio-lateral axis allowing for alignment of a peak point axis of a patella implant along the medio-lateral axis.

In a further embodiment, the clamping element is movable at least along a proximal-distal axis allowing for alignment of the peak point axis of a patella implant along the proximal-distal axis.

In a preferred embodiment, the clamping element is movable both along the medio-lateral axis and along the proximal-distal axis allowing for an alignment of the peak point axis of a patella implant within a plane extending perpendicular to the pressure axis.

In one embodiment, the holding structure comprises at least one guiding element for guiding the clamping element. The guiding element defines at least one axis of movement of the clamping element. For example, the holding structure comprises two distinct guiding elements being arranged parallel to each other.

In one embodiment, the clamping element comprises a guiding slot for sliding engagement of the at least one guiding element. The guiding slot and the at least one guiding element are in a form-fit engagement in a direction parallel to the pressure axis and thus attaching the clamping element to the holding structure in a movable manner.

Preferably, the guiding slot extends along a length which is shorter than a guiding length of the at least one guiding element such that the clamping element is movable along an extension axis of guiding element.

Preferably, the guiding slot has a width which is greater than a width of the at least one guiding element such that the clamping element is movable in a direction perpendicular to an extension axis of guiding element.

In one embodiment, the holding structure has a U-shape which is stable and allows for a compact integration of at least one guiding element. Preferably, the clamping element is made of plastic material to preserve the geometry of a surface of a patella implant during the impaction process. Preferably, the holding structure and/or at least one guiding element are made from metal material.

In a preferred embodiment, the device comprises a second clamp configured to be located on the anterior of the resected patella, and further comprising a pressing device for pressing the first clamp and the second clamp towards each other. The pressing device may be a manual clamp, e.g. of a pincer-type.

In one embodiment, the holding structure of the first clamp and the pressing device are permanently connected to each other allowing for a simple and robust device.

In another embodiment, the holding structure and the pressing device are removably connectable to each other by means of a connector and allowing for the holding structure to be used with different pressing devices or for a pressing device to be used with different holding structures.

The invention also relates to a patella preparation set comprising a patella impaction device as described above and further comprising at least two patella implants, the two patella implants being alternatively being connectable to a resected patella and having identical connection elements for being connecting to the resected patella, wherein the peak point axes of the two patella implants are offset to each other with respect to their medio-lateral axes. Independent of the choice of either one of these two patella implants, the impaction of a selected patella implant and a resected patella can take place along a pressure axis which is aligned with the peak point axis of the selected patella implant.

### BRIEF DESCRIPTION OF THE DRAWINGS

Further features and advantages are the subject of the following description and of the diagrammatic illustration of embodiments.

In the drawings:

FIG. 1 shows a perspective view of one embodiment of a patella impaction device engaging a patella implant;

FIG. 2 shows a sectional view according to sectional plane II-II indicated in FIG. 1;

FIG. 3 shows a side view according to arrow III indicated in FIG. 1;

FIG. 4 shows a rear view of the patella implant of FIG. 1;

FIG. 5 shows a sectional view according to sectional plane V-V indicated in FIG. 4;

FIG. 6 shows a rear view of another patella implant; and

FIG. 7 shows a sectional view according to sectional plane VII-VII indicated in FIG. 6.

### DETAILED DESCRIPTION

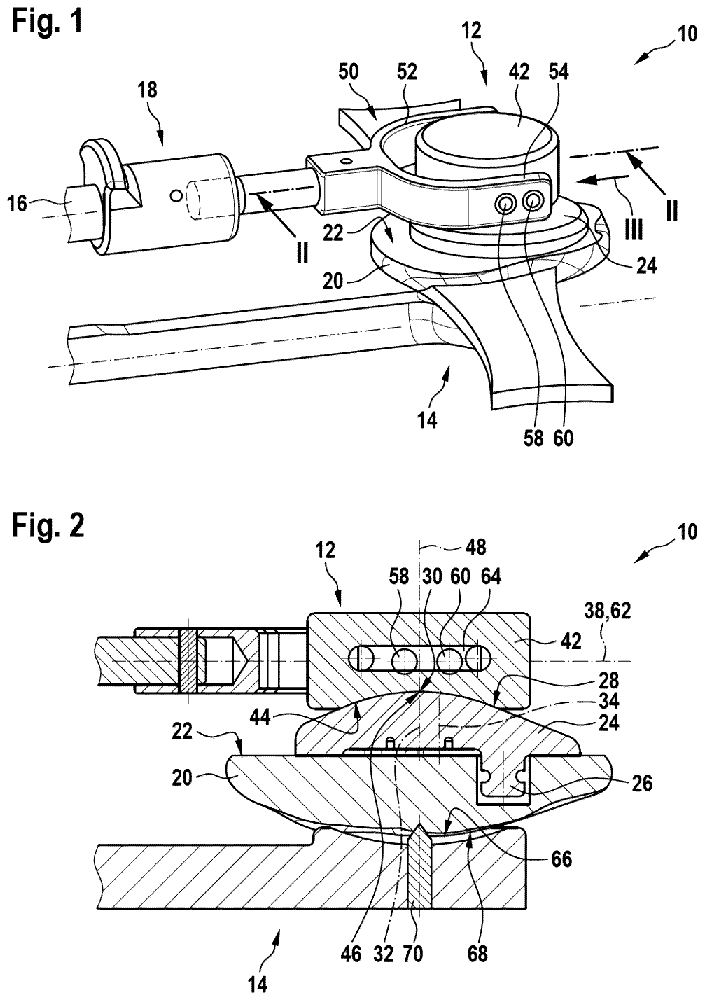

According to FIG. 1, a patella impaction device 10 comprises a first clamp 12 and a second clamp 14 which can be pressed towards each other by means of a pressing device 16. The pressing device is illustrated in a schematic and partial manner only; an example of a pressing device 16 is a clamp with two handles or grips to be manipulated by a hand of a surgeon.

The first clamp 12 is releasably connectable or connected to the pressing device 16 by means of a connector 18 which by way of example may incorporate a threading or snap-fit connection.

The impaction device 10 is used in connection with a resected patella 20 having a resection plane 22 and with a patella implant 24 having connection elements 26 to be connected with the resected patella 20.

By way of example, FIG. 1 shows a right knee patella; a lateral side corresponds with a left side of the drawing and a medial side corresponds with a right side of the drawing. A tibia would be arranged at the front of the drawing and a femur would be arranged in the background of the drawing.

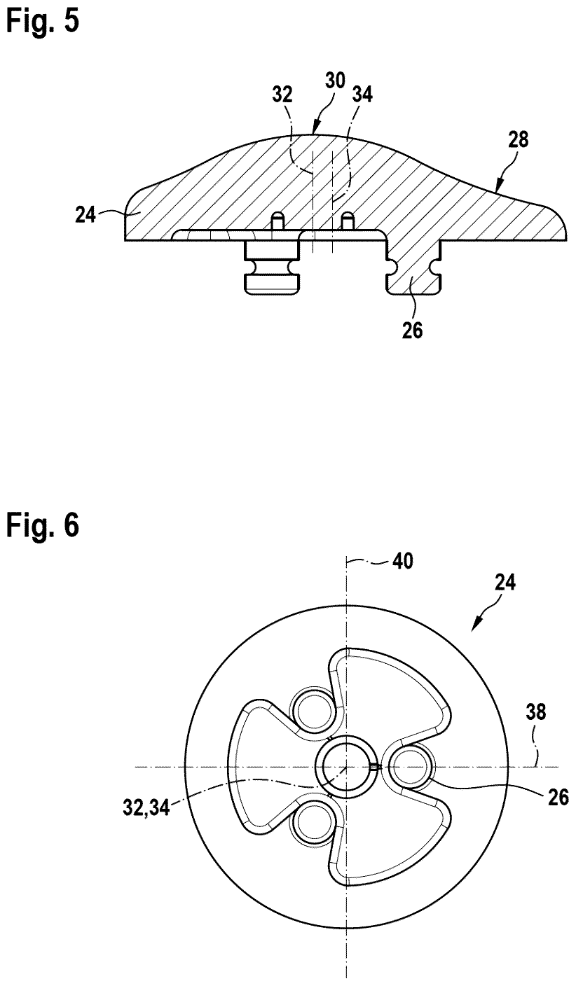

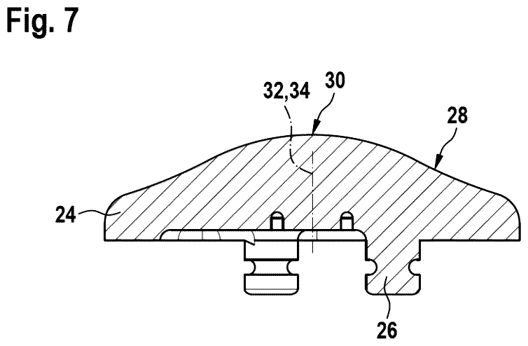

The patella implant 24 comprises a dome-shaped surface 28 which comprises a peak point 30 of a peak point axis 32. In the example shown in FIG. 1 to 5, the peak point axis 32 is offset to a central axis 34 of the patella implant 24. In the example of a patella implant 24 shown in FIGS. 6 and 7, the peak point axis 32 and the central axis 34 are aligned with each other.

The central axis 34 is defined by an intersection point of an extension of the patella implant along a medio-lateral axis 38 and along a proximal-distal axis 40.

The anterior face 28 of the patella implant is dome-shaped and is complementary to a shell-shaped clamping element 42 having a shell 44 with a center or lowest-point 46 which defines a pressure axis 48.

The patella impaction device 10 comprises a U-shaped holding structure 50 with two holding arms 52, 54 extending parallel to each other and delimiting a holding space 56 in which the clamping element 42 is positioned in a movable manner.

The holding arms 52, 54 support two bolt-shaped guiding elements 58, 60 which are arranged within the holding space 56 and which are connected to the holding arms 52, 54. The guiding elements 58, 60 define a plane 62 which is perpendicular to the pressure axis 48. The clamping element 42 is movable within the plane 62 and with respect to the holding structure 50 by means of a guiding slot 64 which surrounds the guiding elements 58, 60.

The guiding slot 64 has a width which is greater than a combined width of the two guiding elements 58, 60, such that the clamping element 42 is movable along a medio-lateral axis 38, see FIG. 2.

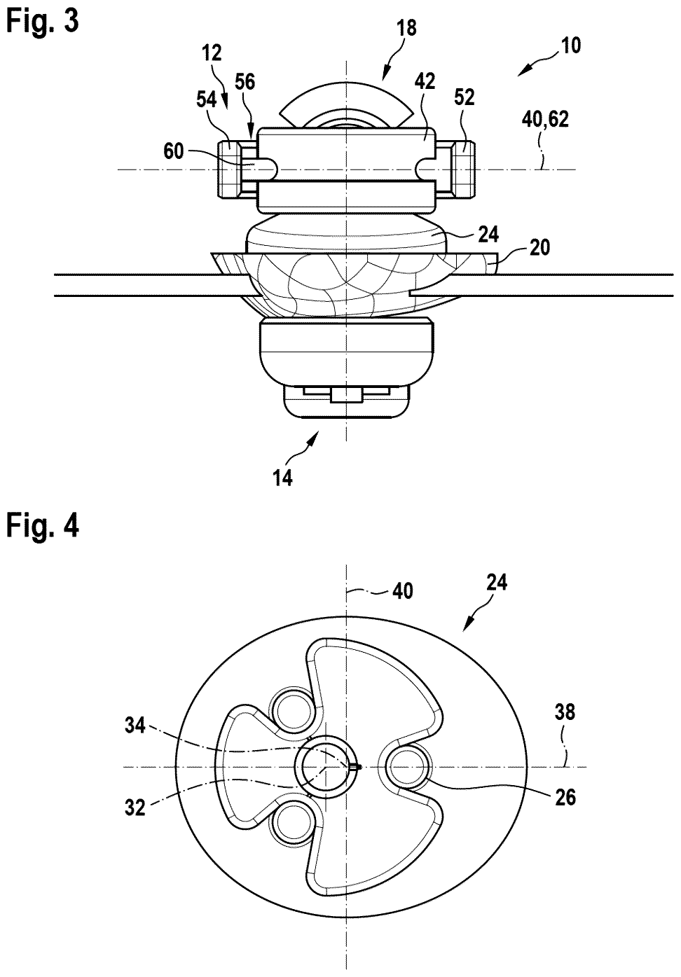

The guiding slot 64 extends along a length which is shorter than a guiding length of the at least one guiding element 58, 60, such that the clamping element 42 is movable along a proximal-distal axis 40, see FIG. 3.

The guiding slot 64 has a width which is greater than a combined width of the two guiding elements 58, 60, such that the clamping element 42 is movable along a medio-lateral axis 38, see FIG. 2.

The second clamp 14 comprises a shell-shaped surface 66 to be pressed against an anterior surface 68 of the resected patella 20. The second clamp 14 may comprise a spike 70 engaging with the surface 68.

When impacting the patella implant 24 to the resected patella 20, the clamping element 42 and its shell 44 are movable within plane 62 in order to self-align the center or lowest-point 46 of the shell 44 and the corresponding pressure axis 48 on the one hand with the peak point 30 of the peak point axis 32 of the patella implant 24 on the other hand.

By way of example, if the patella implant 24 shown in FIGS. 6 and 7 was used in a situation as shown in FIGS. 1, 2 and 3, the peak point axis 32 of the patella implant 24 would be shifted and be aligned with the central axis 34. Assuming that the holding structure 50 remains in the same position as shown in FIGS. 1, 2 and 3, the clamping element 42 would be shifted along the medio-lateral axis 38 accordingly (to the right in FIG. 2).

1. A patella impaction device comprising:
a first clamp configured to be pressed along a pressure axis and onto a dome-shaped surface of a patella implant to be connected to a resected patella; and
a clamping element that is shell-shaped and mounted on a holding structure of the first clamp in a floating manner,
the clamping element being movable at least along one axis of movement within a plane extending perpendicular to the pressure axis.

2. The patella impaction device according to claim 1, wherein the clamping element is movable at least along a medio-lateral axis.

3. The patella impaction device according to claim 1, wherein the clamping element is movable at least along a proximal-distal axis.

4. The patella impaction device according to claim 1, wherein the holding structure comprises at least one guiding element for guiding the clamping element.

5. The patella impaction device according to claim 4, wherein the at least one guiding element comprises a first guiding element and a second guiding element arranged parallel to the first guiding element.

6. The patella impaction device according to claim 4, wherein the clamping element comprises a guiding slot for sliding engagement of the at least one guiding element.

7. The patella impaction device according to claim 6, wherein the guiding slot extends along a length that is shorter than a guiding length of the at least one guiding element and/or the guiding slot has a width that is greater than a width of the at least one guiding element.

8. The patella impaction device according to claim 1, wherein the holding structure has a U-shape.

9. The patella impaction device according to claim 1, wherein the clamping element is made of plastic material.

10. The patella impaction device according to claim 1, further comprising:
a second clamp configured to be located on an anterior region of the resected patella; and
a pressing device for pressing the first clamp and the second clamp towards each other.

11. The patella impaction device according to claim 10, wherein the holding structure of the first clamp and the pressing device are permanently connected to each other and/or the holding structure and the pressing device are removably connectable to each other by a connector.

12. A patella preparation set comprising:
a patella impaction device according to claim 1; and
at least two patella implants that are alternatively connectable to a resected patella,
the at least two patella implants having identical connection elements for connecting to the resected patella, and
the at least two patella implants having peak point axes that are offset to each other with respect to their medio-lateral axes.
